(19) United States
(12) Patent Application Publication
(10) Pub. No.: US 2007/0010436 A1
(43) Pub. Date: Jan. 11, 2007

(54) Remedy for cardiomyopathy

(76) Inventors: Yasuhiko Tabata, Kyoto (JP); Masashi Komeda, Kyoto (JP)

Correspondence Address: OSHA LIANG L.L.P., 1221 MCKINNEY STREET, SUITE 2800, HOUSTON, TX, 77010, US

(21) Appl. No.: 10/551,497

(22) PCT Filed: Apr. 1, 2003
(86) PCT No.: PCT/JP03/04164

§ 371 Date: Jul. 27, 2006

(51) Int. Cl. A61K 38/17; A61K 38/39

(52) U.S. Cl. 514/12

(57) ABSTRACT

The present invention provides a cardiomyopathy therapeutic agent that contains hepatocyte growth factor (HGF) and gelatin hydrogel, and gradually releases HGF, which is useful in treating cardiomyopathy.

### TECHNICAL FIELD

The present invention relates to cardiomyopathy therapeutic agent that contains hepatocyte growth factor (HGF) and gelatin hydrogel, and gradually releases HGF.

### BACKGROUND ART

Hepatocyte growth factor (HGF) is a growth factor that was partially purified from rat blood during liver regeneration as a growth factor for mature rat primary cultured hepatocytes by Nakamura et al. in 1984, and its gene has been cloned (Biochem. Biophys. Res. Commun., 122, 1450 (1984); Proc. Natl. Acad. Sci. USA, 83, 6489 (1986); FEBS Letters, 22, 311 (1987); Nature, 342, 440 (1989); Proc. Natl. Acad. Sci. USA, 87, 3200 (1990)).

As a result of subsequent research, HGF was determined to not only act to promote growth by functioning to repair and regenerate liver damage as a liver regeneration factor in vitro, but also have extremely diverse properties including promoting migration with respect to various target cells, inducing morphogenesis and inhibiting apoptosis, thereby play an important role as a regeneration and maintenance factor of organs and tissues, and with respect to the heart, have cardiovascular protective action such as promotion of vascularization, prevention of reperfusion injury and inhibition of fibrosis, thereby play an important role in the treatment or prevention of ischemic diseases and artery diseases (Symp. Soc. Exp. Biol., 47, Cell Behavior, 227-234 (1993); Proc. Natl. Acad. Sci. USA, 90, 1937-1941 (1993); Circulation, 97, 381-390 (1998)).

In this manner, HGF has various functions including vascularization action. Consequently, various attempts have been made to use HGF as a pharmaceutical.

However, since the half-life of HGF in the blood is short at several to ten minutes, it is difficult to maintain its concentration in the blood. In addition, there was also the problem of inadequate transmigration of HGF to an affected area. Thus, if HGF is merely administered as an aqueous solution, it ends up rapidly diffusing from the administration site and subsequently being excreted, thereby making it difficult to obtain adequate effects of the physiological activity of HGF.

A treatment method for cardiomyopathy that uses HGF gene has also been developed. This treatment method consists of administering the gene mainly into muscle, causing the gene to be incorporated in muscle cells, and causing the expression product of the inserted gene in the form of protein to be secreted from the cells containing the gene. This method is characterized by gradual release using cells, or in other words, causing cells to gradually release a vascularization induction factor. However, this method has the shortcomings of the gene expression efficiency of HGF being low, and being unable to control the level or timing of gene expression. In addition, there is also the problem of the expression of unknown effects resulting from gene insertion still not having been resolved.

In brief, the most important factor in terms of solving the aforementioned problems is the gradual release of vascularization induction factor. The reason for attempting to secrete a cell growth factor from cells using a gene and obtain the effects of its gradual release is that, in the case of administering a vascularization induction factor in the form of an aqueous solution, expression of the action of the vascularization induction factor is not observed at all, and the vascularization induction factor itself is unable to be gradually released.

If it were possible to gradually release a cell growth factor as in the present invention however, it would be meaningless to select a method that uses a gene, and the aforementioned problems would be able to be solved.

The only way to increase efficacy in vivo is to immerse HGF in a polymer carrier to enable gradual release of HGF over a long period of time. In recent years, several tests have shown that, in the case of combining the use of various carrier matrices, several growth factors such as basic fibroblast growth factor, bone morphogenic protein and transforming growth factor demonstrate predicted physiological activity in vivo (Downs, E. C. et al., 1992; Miyamoto, et al., 1992; Gombotx, W. R. et al., 1993). However, there are no reports whatsoever regarding the gradual release of HGF in vivo. There have only been several research results which have shown that predicted physiological activity can be induced when a physiologically excess dose of HGF solution is injected.

In addition, HGF is known to inhibit fibrosis of heart muscle by means of blocking angiotensin II in laboratory animals (Taniyama, T. et al., Circulation (2000) 102: 246-252). It is also conversely known that impairment of HGF production in heart failure patients can be eliminated by administration of angiotensin convertase inhibitor (Yasuda, S. et al., Hypertension (1993) 33:1374-1378).

On the other hand, although dilated cardiomyopathy is a refractory disease characterized by fibrosis of heart muscle and its accompanying degeneration (hypertrophy, atrophy) of heart muscle cells, an effective treatment method has yet to be found.

As a result of conducting extensive studies on therapeutic agents for dilated cardiomyopathy, the inventors of the present invention found that treatment using an HGF gradual release preparation that uses a gelatin hydrogel developed by Tabata et al. demonstrates remarkable therapeutic effects against heart disease in dilated cardiomyopathy model rats, thereby leading to completion of the present invention.

### DISCLOSURE OF THE INVENTION

The object of the present invention is to provide a cardiomyopathy therapeutic agent that contains HGF and gelatin hydrogel, and gradually releases HGF.

A gelatin used in the present invention differs from commercially available gelatin and is a gelatin that has the following physical properties:
(1)an acidic gelatin obtained from collagen by alkaline hydrolysis treatment;
(2)molecular weight under non-reducing conditions of SDS-PAGE of about 100,000 to about 200,000 daltons; and,
(3)the zeta potential in aqueous solution of about −15 to about −20 mV.

Although examples of commercially available gelatins include the type A gelatin manufactured by Sigma and the gelatin manufactured by Wako Pure Chemical Industries, the zeta potential in aqueous solution differs in the manner shown below.

Sigma Type A Gelatin: Roughly 0 to roughly 5 mV

Wako Gelatin: Roughly −5 to roughly −2 mV

The zeta potential is an indicator that represents the degree of electrostatic charge of a substance (gelatin), and is considered to be suitable as an indicator of a gelatin that forms an electrostatic complex with HGF in the present invention.

A gelatin of the present invention is obtained by alkaline hydrolysis from a part such as the skin or tendon of various animal species such as cows, from collagen, or from a substance used as collagen. Preferably, it is an acidic gelatin prepared by alkaline treatment of type I collagen originating in bovine bone, and can also be acquired having a sample isoelectric point (IEP) of 5.0 from Nitta Gelatin. Furthermore, although basic gelatin prepared by acid treatment can also be similarly acquired from Nitta Gelatin having an IEP of 9.0, the zeta potential is considerably different as indicated below.

Acidic gelatin (Nitta Gelatin sample IEP 5.0): Roughly −15 to roughly −20 mV

Basic gelatin (Nitta Gelatin sample IEP 9.0): Roughly +12 to roughly +15 mV

A gelatin hydrogel used in the present invention refers to a hydrogel obtained by using the aforementioned gelatin and condensing with various chemical crosslinking agents. Examples of chemical crosslinking agents that can be used include glutaraldehyde, EDC and other water-soluble carbodiimides, propylene oxide, diepoxy compounds and condensation agents. An example of a chemical crosslinking agent that is used preferably is glutaraldehyde.

In addition, the gelatin can also be crosslinked by heat treatment or ultraviolet irradiation.

There are no particular limitations on the form of the gelatin hydrogel, and examples include cylinders, square columns, sheets, disks, spheres and particles. Gelatin hydrogels in the form of cylinders, square columns, sheets and disks are frequently used as implants, while spheres and particles can also be administered by injection.

Gelatin hydrogels in the form of cylinders, square columns, sheets and disks can be prepared by adding a crosslinking agent aqueous solution to a gelatin aqueous solution or adding gelatin to a crosslinking agent aqueous solution, followed by pouring into a mold of a desired shape and allowing the crosslinking reaction to proceed. In addition, a molded gelatin gel may be added directly or after drying to a crosslinking agent aqueous solution. The crosslinking reaction is stopped by contacting with a low molecular weight substance having an amino group such as ethanol amine or glycine, or by adding an aqueous solution having a pH of 2.5 or lower. The resulting gelatin hydrogel is used to prepare a preparation after washing with distilled water, ethanol, 2-propanol or acetone and so forth.

A gelatin hydrogel in the form of spheres or particles can be prepared by, for example, attaching an immobilized stirring motor (for example, the 3-1 motor, EYELA mini D.C. stirrer, manufactured by Shinto Scientific) and Teflon (registered trademark) propeller to a three-mouth, round-bottom flask, placing the flask and immobilized apparatus in a gelatin solution, adding an oil such as olive oil, stirring at a speed of about 200 to 600 rpm to form a W/O emulsion and adding a crosslinking agent aqueous solution thereto, or dropping the product of pre-emulsifying a gelatin aqueous solution in olive oil (for example, using the Advantec 21 vortex mixer, homogenizer or polytron PT10-35) into olive oil to prepare a fine particulate W/O emulsion, followed by the addition of a crosslinking agent aqueous solution and allowing the crosslinking reaction to proceed. After then recovering the gelatin hydrogel by centrifugal separation, it is washed with ethyl acetate and so forth followed by immersing in 2-propanol or ethanol to stop the crosslinking reaction. The resulting gelatin hydrogen particles are used to prepare a preparation after sequentially washing with 2-propanol, Tween 80-containing distilled water and distilled water.

In the case the gelatin hydrogel particles aggregate, the addition of, for example, a surfactant or ultrasonic treatment (preferably for no more than about 1 minute while cooling) may be carried out.

Furthermore, a fine particulate gelatin hydrogel having a particle size of 20 μm or less can be obtained by pre-emulsification.

The mean particle size of the resulting gelatin hydrogel particles is 1 to 1000 μm, and these particles should be used after sizing to the required size according to the purpose of use.

The following provides an example of another method of preparing a gelatin hydrogel in the form of spheres or particles.

After placing olive oil in the same apparatus as used in the previous example, stirring at a speed of about 200 to 600 rpm, dropping in a gelatin aqueous solution to prepare a W/O emulsion and cooling, ethyl acetate and so forth is added and stirred followed by recovering the gelatin particles by centrifugal separation. After additionally washing the recovered gelatin particles with acetone and ethyl acetate, and then with 2-propanol and ethanol, etc., the particles are allowed to dry. The dry gelatin particles are then suspended in a crosslinking agent aqueous solution containing 0.1% Tween 80, the crosslinking reaction is allowed to proceed while stirring gently, and the particles are washed with 100 mM glycine aqueous solution containing 0.1% Tween 80 or 0.004 N HCl containing 0.1% Tween 80 depending on the crosslinking agent used followed by stopping the crosslinking reaction to prepare gelatin hydrogel particles. The mean particle size of the gelatin hydrogel particles obtained with this method is similar to that in the case of the aforementioned method.

The mechanism of this gradual release is based on vascularization induction factor being physically immobilized on gelatin within the hydrogel. In this state, the factor is not released from the hydrogel. If the gelatin molecules become soluble in water as a result of the hydrogel being decomposed, the immobilized vascularization induction factor is released accompanying that decomposition. Namely, the gradual release properties of the vascularization induction factor can be controlled by the decomposition of the hydrogel. The ease of decomposition of the hydrogel can be changed according to the degree of crosslinking during preparation of the hydrogel.

There are no particular limitations on the conditions of the crosslinking reaction, and it can be carried out, for example, at 0 to 40° C. for 1 to 48 hours.

A gelatin hydrogel of the present invention is such that its moisture content clearly has a considerable effect on the gradual release properties of the vascularization induction factor, and an example of a moisture content that demonstrates preferable gradual release effects is about 80 to 99 w/w %, while a more preferable moisture content is about 95 to 98 w/w %. Moisture content can be used as a measurable indicator of the degree of crosslinking. A large moisture content indicates a low degree of crosslinking as well as greater susceptibility to decomposition. In other words, the value of this moisture content affects the gradual release properties of the vascularization induction factor.

A gelatin hydrogel of the present invention can be used after suitably cutting to an appropriate size and shape, freeze-drying and sterilizing. Freeze-drying can be carried out by, for example, placing the gelatin hydrogel in distilled water, freezing for 30 minutes or more in liquid nitrogen or for 1 hour or more at −80° C., and then drying for 1 to 3 days in a freeze-dryer.

Although the concentrations of gelatin and crosslinking agent when preparing a gelatin hydrogel should be suitably selected according to the desired moisture content, an example of the gelatin concentration is 1 to 20 w/w %, while an example of the crosslinking agent concentration is 0.01 to 1 w/w %.

The HGF used in the present invention is a known substance, and that prepared by various methods can be used provided it has been purified to a degree that allows it to be used as a pharmaceutical. In addition, a commercially available product (such as Toyobo Code No. HGF-101) may also be used. An example of a method for producing HGF consists of culturing primary cultured cells or established cells that produce HGF, separating from the culture supernatant and so forth, and purifying to obtain said HGF. Alternatively, a gene that encodes HGF can be incorporated in a suitable vector using genetic engineering techniques followed by transformation of a suitable host by inserting in said host, and then obtaining the target recombinant HGF from the culture supernatant of the transformants (refer to, for example, Nature, 342, 440 (1989); Japanese Unexamined Patent Publication No. JP1993-111382; Biochem. Biophys. Res. Commun. 163, 967 (1989)). There are no particular limitations on the aforementioned host cells, and various host cells conventionally used in genetic engineering techniques can be used, examples of which include E. coli, yeast and animal cells. The HGF obtained in this manner may have one or multiple amino acids in its amino acid sequence substituted, deleted and/or added, or a sugar chain may be similarly substituted, deleted and/or added, provided it has substantially the same action as naturally-occurring HGF.

An HGF gradual release gelatin hydrogel preparation in the present invention refers to a preparation that is obtained by immersing HGF into the aforementioned acidic gelatin hydrogel. Although HGF forms a complex with acidic gelatin hydrogel because it is a basic protein, when considering the absorption inhibitory effects of HGF with respect to the aforementioned changes in ionic strength in solution, this HGF gelatin (hydrogel) complex not only involves electrostatic interaction, but is also affected by other interactions such as hydrophobic bonding. The dissociation constant (Kd) of this complex as well as the binding molar ratio of HGF to gelatin were obtained according to a Scatchard binding model (Scatchard, G., 1949). The binding molar ratio of HGF to gelatin is such that roughly 7 HGF molecules bind to 1 acidic gelatin molecule.

In addition, the Kd value of acidic gelatin at 37° C. is 5.5×10⁻⁷ M, which is about two to three orders larger than the Kd value of heparin sulfate at 20° C. of 1×10⁻⁹ to 2.0×10⁻¹⁰ M (Rahmoune, H. et al., 1988). This indicates that binding of the HGF gelatin complex is weak and not as strong as that between HGF and heparin sulfate.

In the case the molar ratio of HGF to gelatin is about 1:7 or more, liberation of HGF occurs easily and the resulting behavior is quite similar to that of free HGF in terms of activity. However, in the case the molar ratio of HGF to gelatin is lowered to about 1:7 or less, since the HGF is adsorbed and the amount that is liberated is reduced, the apparent activity of HGF appears to decrease.

Thus, although a complex of HGF and gelatin or gelatin hydrogel can be made in which the molar ratio between the HGF and gelatin is changed in various ways, in order to avoid an initial burst, a preferable example of a complex has a molar ratio in which there are about 7 moles or less of HGF to 1 mole of gelatin hydrogel.

Furthermore, the weight ratio of HGF to gelatin is preferably amount 5 or less, and the weight ratio of HGF to gelatin is more preferably about 5 to about 1/10⁴.

Since an HGF gradual release gelatin hydrogel preparation of the present invention has HGF gradual release effects and HGF stabilizing effects, it is able to demonstrate the function of HGF for a long period of time even in small amounts. Consequently, the inherent function of HGF in the form of cardiovascular protective action such as promotion of vascularization, prevention of reperfusion injury and inhibition of fibrosis are demonstrated, thereby enabling it to be effectively used as a cardiomyopathy therapeutic agent.

An HGF gelatin hydrogel preparation of the present invention can be used parenterally as an injection preparation. It can be administered, for example, subcutaneously, intramuscularly, intravenously, intracelomicly, into connective tissue, intraperiosteally or into a damaged organ.

An HGF gradual release gelatin hydrogel preparation of the present invention or complex thereof can be used in a suitable drug form according to the respective application. For example, it can be administered in a drug form such as a sheet, stick, particles, rods or paste. Examples of administration methods include intracutaneous, subcutaneous, intramuscular, intracelomic, into connective tissue and intraperiosteal administration.

Although the dosage of HGF in a preparation of the present invention can be suitably adjusted according to the patient's severity, patient's age and body weight, etc., the normal adult dosage is selected from the range of about 0.01 to about 5 μg, and preferably from the range of about 0.01 to about 0.5 μg, and can be injected into the affected area of a peripheral site thereof. In addition, said administration can be performed a plurality of times in the case effects are inadequate with a single administration.

As previously described, the applicable disease of an HGF gradual release gelatin hydrogel preparation of the present invention is cardiomyopathy. Cardiomyopathy as referred to in the present invention refers to all diseases for which lesions are observed in heart muscle that are characterized by the absence of a well-defined cause and abnormal hypertrophy, degeneration or fibrosis of heart muscle.

Specific examples of applicable diseases include dilated cardiomyopathy or hypertrophic cardiomyopathy, idiopathic cardiomyopathy, primary cardiomyopathy and secondary cardiomyopathy, while dilated cardiomyopathy is preferable. With respect to secondary cardiomyopathy, secondary cardiomyopathy accompanied by adverse drug side effects, toxin action or viral or bacterial infection is preferable.

### BRIEF DESCRIPTION OF THE DRAWINGS

FIG. 1 is a graph showing changes in left ventricular telediastolic diameter. In contrast to coronary dilation being not only inhibited, but conversely reduced in an HGF treatment group, coronary dilation was observed in a sham group.

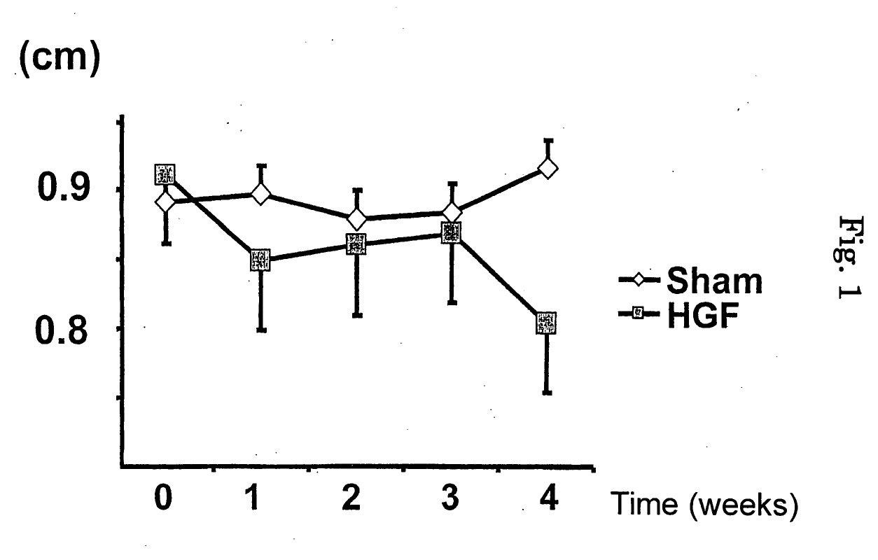

FIG. 2 is a graph showing changes in left ventricular telesystolic diameter. In contrast to coronary dilation being not only inhibited, but conversely reduced in an HGF treatment group, coronary dilation was observed in a sham group.

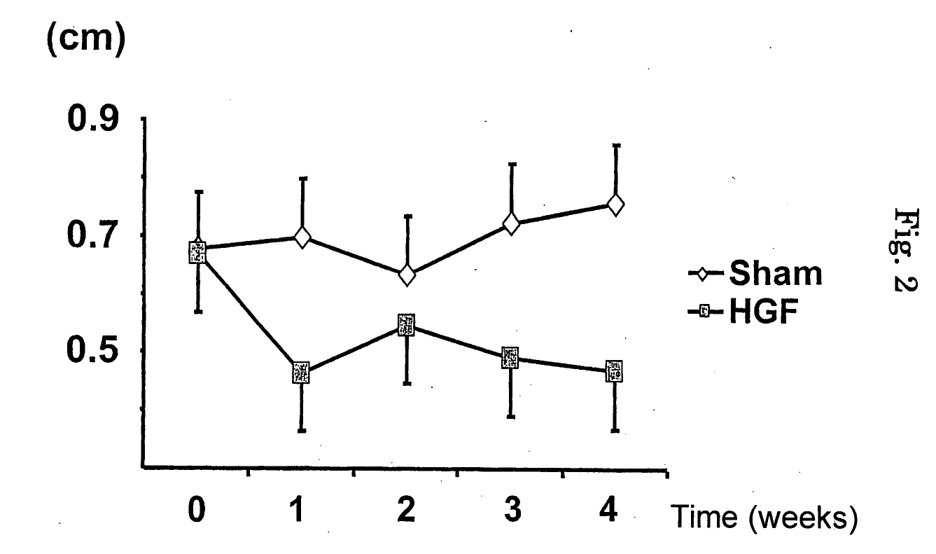

FIG. 3 is a graph showing changes in left ventricular minor axis shortening rate (rate of shortening of the left ventricular diameter that occurs accompanying contraction in a circular cross-section of the heart). In contrast to improvement of coronary systole being observed in an HGF treatment group, exacerbation of coronary function progressed in a sham group.

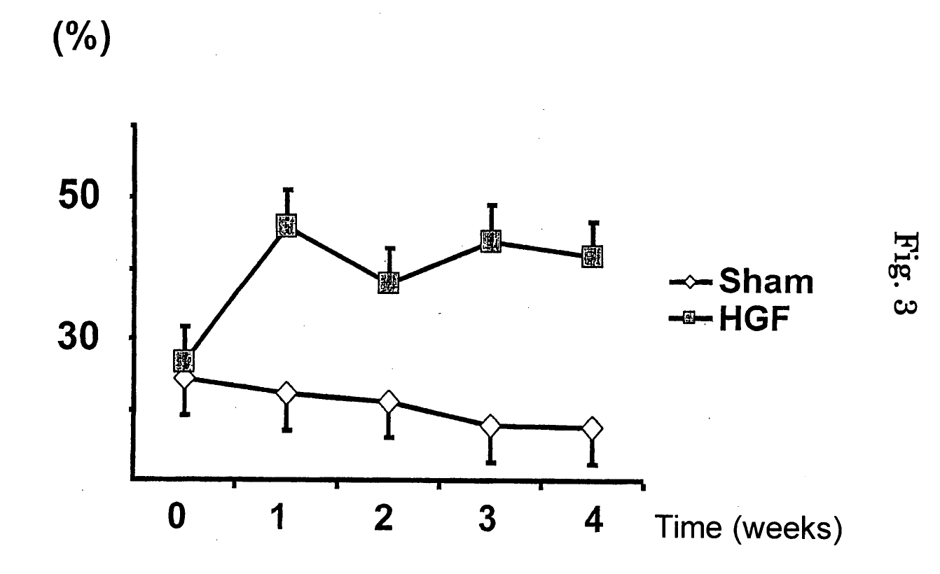

FIG. 4 is a graph showing changes in the rate of change of left ventricular lumen surface area (rate of reduction of left ventricular cross-sectional area that occurs accompanying contraction in a circular cross-section of the heart). In contrast to improvement of coronary systole being observed in an HGF treatment group, exacerbation of coronary function progressed in a sham group.

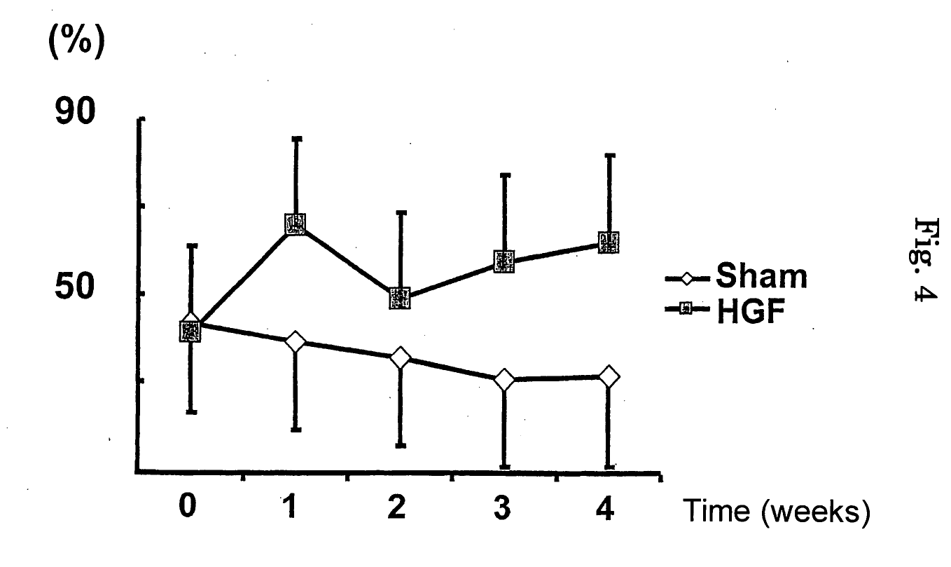

### EXAMPLES

Experimental Method

An HGF gradual release agent was prepared according to the method of Tabata et al. so that the gradual release of HGF continued for about 4 hours after administration. A dilated cardiomyopathy model was prepared by inducing acute myocarditis by subcutaneously administering myosin derived from porcine heart muscle to Lewis rats (males, n =9, purchased from Shimizu Laboratory Animals) followed by allowing six weeks to elapse to induce cardiomyopathy. These animals were divided into an HGF treatment group (n =4) and a sham group (n=5). A gelatin sheet immersed with HGF gradual release agent was affixed to the left ventricular anterior wall of the animals of the HGF treatment group following thoracotomy to promote subsequent gradual release of HGF, while a gelatin sheet immersed with saline was adhered to the left ventricular anterior wall of the animals of the sham group. The size and function of the heart were followed up for 4 weeks after surgery by echocardiography using an ultrasonic probe at a frequency of 10 to 12 MHz.

Experimental Results

In contrast to coronary dilation being not only inhibited, but conversely reduced in the HGF treatment group, coronary dilation was observed in the sham group.

Left ventricular telediastolic diameter (cm):

| | Preoperative | After 2 weeks | After 4 weeks |
|---|---|---|---|
| HGF group | 0.91 ± 0.04 | 0.86 ± 0.05 | 0.80 ± 0.05 |
| Sham group | 0.89 ± 0.03 | 0.88 ± 0.03 | 0.91 ± 0.05* |

*p = 0.0043

Left ventricular telesystolic diameter (cm):

| | Preoperative | After 2 weeks | After 4 weeks |
|---|---|---|---|
| HGF group | 0.67 ± 0.02 | 0.55 ± 0.08 | 0.47 ± 0.07 |
| Sham group | 0.68 ± 0.01 | 0.63 ± 0.06 | 0.74 ± 0.05* |

*p = 0.0011

In addition, in contrast to improvement of coronary systole being observed in the HGF treatment group, exacerbation of coronary function progressed in the sham group.

Left ventricular minor axis shortening rate (%)

| | Preoperative | After 2 weeks | After 4 weeks |
|---|---|---|---|
| HGF group | 26.7 ± 1.9 | 37.9 ± 3.4 | 41.7 ± 9.3 |
| Sham group | 24.3 ± 2.2 | 21.1 ± 8.7 | 17.0 ± 2.8* |

*p = 0.0014

Left ventricular lumen surface area change rate (%)

| | Preoperative | After 2 weeks | After 4 weeks |
|---|---|---|---|
| HGF group | 40.8 ± 5.2 | 48.7 ± 11.2 | 61.8 ± 14.9 |
| Sham group | 43.1 ± 3.9 | 35.0 ± 8.0 | 30.3 ± 3.3* |

*p = 0.0010

Direct administration of HGF gradual release agent to heart muscle resulted in a remarkable improvement of coronary contraction and coronary systole for 4 weeks after surgery. This technique was suggested to not only inhibit progression of dilation cardiomyopathy, but also demonstrate aggressive therapeutic effects.

1. A cardiomyopathy therapeutic agent that contains hepatocyte growth factor (HGF) and gelatin hydrogel, and gradually releases HGF.

2. A cardiomyopathy therapeutic agent according to claim 1 wherein the gelatin has the following physical properties:
(1) an acidic gelatin obtained from collagen by alkaline hydrolysis treatment;
(2) molecular weight under non-reducing conditions of SDS-PAGE of about 100,000 to about 200,000 daltons; and;
(3) zeta potential in aqueous solution of about −15 to about −020 mV.

3. A cardiomyopathy therapeutic agent according to claim 1 wherein cardiomyopathy is dilation cardiomyopathy.

4. A cardiomyopathy therapeutic agent according to claim 1 wherein the cardiomyopathy is hypertrophic cardiomyopathy.

5. A cardiomyopathy therapeutic agent according to claim 3 wherein the cardiomyopathy is idiopathic cardiomyopathy, primary cardiomyopathy or secondary cardiomyopathy.

6. A cardiomyopathy therapeutic agent according to claim 2 wherein cardiomyopathy is dilation cardiomyopathy.

7. A cardiomyopathy therapeutic agent according to claim 2 wherein the cardiomyopathy is hypertrophic cardiomyopathy.

8. A cardiomyopathy therapeutic agent according to claim 6 wherein the cardiomyopathy is idiopathic cardiomyopathy, primary cardiomyopathy or secondary cardiomyopathy.

9. A cardiomyopathy therapeutic agent according to claim 4 wherein the cardiomyopathy is idiopathic cardiomyopathy, primary cardiomyopathy or secondary omyopathy.

10. A cardiomyopathy therapeutic agent according to claim 7 wherein the cardiomyopathy is idiopathic cardiomyopathy, primary cardiomyopathy or secondary cardiomyopathy.
